(19) United States
(12) Patent Application Publication
(10) Pub. No.: US 2010/0198235 A1
(43) Pub. Date: Aug. 5, 2010

(54) SURGICAL SUTURE PASSER AND METHOD FOR PASSING SUTURE

(76) Inventors: Javin Cedric Pierce, Stowe, VT (US); Stephen Williams, Montpelier, VT (US)

Correspondence Address: Mark J. Pandiscio;Pandiscio & Pandiscio, P.C., 470 Totten Pond Road, Waltham, MA, 02451, US

(21) Appl. No.: 12/569,576

(22) Filed: Sep. 29, 2009

Related U.S. Application Data
(60) Provisional application No. 61/100,821, filed on Sep. 29, 2008.

(51) Int. Cl. A61B 17/04

(52) U.S. Cl. 606/148

(57) ABSTRACT

A suture placement system that serves as both an open or endoscopic surgical repair means wherein tissue defects can be externally ligated in close opposition by the steps of: passing a sharp penetrating needle through tissue, wherein the sharp penetrating needle carries a suture near the distal end of the needle; capturing a portion of the suture with a snare that is mounted to an axially and rotationally mobile armature that is carried by the needle; and withdrawing a portion of the suture which has been passed through the tissue which is to be repaired. And a suture placement system that serves as both an open or endoscopic surgical repair means wherein tissue defects can be externally ligated in close opposition by the steps of: passing a sharp penetrating needle comprising a shaft and handle through tissue, wherein the sharp penetrating needle carries a suture snare near the distal end of the needle; positioning a suture adjacent to the suture snare using a cannula that is mounted to an axially and rotationally mobile armature that is carried by the needle; capturing a portion of the suture with the suture snare; and withdrawing a portion of the suture through the tissue which is to be repaired.

### REFERENCE TO PENDING PRIOR PATENT APPLICATION

This patent application claims benefit of pending prior U.S. Provisional Patent Application Ser. No. 61/100,821, filed Sep. 29, 2008 by Javin Cedric Pierce et al. for SURGICAL SUTURE PASSER APPARATUS AND METHOD (Attorney's Docket No. PIERCE-1 PROV), which patent application is hereby incorporated herein by reference.

### FIELD OF THE INVENTION

This invention relates to suture placement and suturing in general, and more particularly to a surgical suture passer where one element is used to carry suture and another element is used to capture suture, whereby to pass suture through tissue.

### BACKGROUND OF THE INVENTION

Numerous and various types of suture placement devices are known in the art for facilitating the repair of tissue by way of passing surgical sutures, but many of these prior art devices are limited by their inability to both pass and retrieve sutures using the same instrument.

### Devices for Only Passing Suture

Various devices are designed to pass suture. Many of these devices are designed to only pass suture and not to retrieve suture. By way of example but not limitation, the Conmed Linvatec Spectrum and Spectrum II suture passers comprise a sharp curved cannula designed to pass suture down the interior lumen of the cannula and then out of the distal portion of the curved sharp cannula. The Spectrum II Roller Wheel facilitates the passing of suture by providing a means to advance suture though the curved sharp cannula.

U.S. Patent Application Publication No. 2005/0283171 also discloses an embodiment of a sharp curved cannula with an integrated roller wheel.

U.S. Patent Application Publication No. 2007/0179510 discloses a suture passing device comprising a sharp curved cannula with an integrated suture advancement mechanism.

The Arthrex AC Caracoid Graft Passing Instrument comprises an elongated cannula with a hollow, corkscrew-shaped tip for passing suture.

The Arthrex Pigtail product comprises a curved structure for passing suture.

However, none of the foregoing suture passing devices provides, or suggests the provision of, an integrated suture retrieving means.

Other suture passers also exist that can pass suture through tissue, such as laterally-curved suture hooks, however, all of these suture passing instruments require a secondary instrument to retrieve the suture and draw it through the tissue so it can be used to form a ligature.

### Devices for Only Retrieving Suture

In addition to the foregoing, suture retrieval devices are known in the art for retrieving suture. Many of these devices are designed to only retrieve suture and not to pass suture. In some cases, the suture retrieval device may also include a sharp needle to penetrate tissue so as to position the suture retrieval mechanism on the far side of the tissue, but does not provide an integrated suture passing means.

By way of example but not limitation, U.S. Pat. No. 6,991,636 discloses a sharp curved cannula with a wire loop suture retrieval means.

Conmed Linvatec markets the Blitz Suture Retriever. The Blitz device comprises a sharp curved cannula through which a suture-retrieving Nitinol wire loop is deployed after the sharp cannula has penetrated through to the far side of tissue. The wire loop captures the suture and then pulls the suture back through the tissue.

However, these suture retrieval devices do not provide, or suggest the provision of, an integrated mechanism for passing the suture, and hence these suture retrieval devices still require the use of a second instrument for the initial passing of suture.

### Integrated Suture Passing and Retrieval Devices

Still other devices may provide integrated suture passing and retrieval means. See, for example, U.S. Pat. Nos. D343,728; 5,522,820; 4,923,461; and 4,890,615. However, these integrated suture passing and retrieval devices are generally limited to linear suture passing and retrieval motions.

U.S. Pat. No. 5,643,292 discloses a suture passing instrument providing an integrated suture passing and retrieval means. More particularly, a curved sharp cannula is used to penetrate tissue. Suture is passed through the curved sharp cannula, with suture passage facilitated by the use of an integrated thumbwheel. A suture retrieval snare is passed through the same curved sharp cannula and is used to capture the suture and draw it back into the curved sharp cannula. This instrument, however, does not provide the simultaneous disposition of separate suture passing means and suture retrieval means on the far side of the tissue, and does not provide suture passing means and suture retrieval means wherein one or the other moves both axially and rotationally relative to the other.

The Conmed Linvatec Spectrum MVP Suture Passer comprises a curved sharp cannula and a second, integrated hollow straight cannula. The curved sharp cannula is designed to pass a suture retrieval wire, whereas the hollow straight cannula is used to pass a suture by means of a suture-carrying device. The suture-carrying device presumably passes the suture into the open eye of the retrieval wire, which captures the suture and pulls it back into the curved sharp cannula. While this device provides integrated suture passing means and suture retrieval means, it does not provide suture passing means and suture retrieval means wherein one or the other moves both axially and rotationally relative to the other.

### Other Instruments

Arthrex markets a number of arthroscopic instruments comprising hollow sharp cannulas with curved tips through which a Nitinol or other wire loop suture retriever may be passed. These Arthrex instruments include instruments marketed as SutureLasso, SutureLassoSD and Micro SutureLasso. Some of these instruments may also be used to pass suture through the sharp cannula. However, all of these instruments comprise a single lumen cannula and do not provide the combination of suture passing and suture retrieval integrated into one instrument. These instruments can either pass suture or the wire loop, but not both, in one procedure.

The prior art suture placing apparatus and suturing procedures present many problems to the medical professional and to the patient. More particularly, where separate instruments are used to pass and retrieve suture, at least two instruments (i.e., one to pass and one to retrieve) are needed, and the suture is often difficult to capture in free space. Another drawback relates to the time spent, and the difficulty in properly positioning, the two separate instruments within the joint. Furthermore, in endoscopic procedures, the need for a second instrument necessitates the use of a second access portal and another hand. And in such endoscopic procedures, the need for a second instrument portal can displace the visualization portal, so that the means to view the procedure may be restricted. Furthermore, a third step is often required in order to shuttle suture back out the first instrument portal.

Even where prior art systems provide a single integrated instrument for passing and retrieving suture, these instruments are limited in their function and performance.

Thus, there is a need in the art for a new and improved apparatus and method for surgically passing and retrieving suture in a manner that is safe, reliable, user-friendly and effective.

### SUMMARY OF THE PRESENT INVENTION

The present invention comprises the provision and use of a novel surgical suture passer which is capable of both delivering and retrieving suture at the surgical site.

In one form of the invention, there is provided a novel surgical suture passer which generally comprises an elongated shaft having a proximal end and a distal end. The distal end of the elongated shaft comprises a suture passing mechanism for passing suture through the elongated shaft and out of the distal end of the suture passing mechanism. The distal end of the elongated shaft further comprises a suture retrieving mechanism for retrieving the suture extending from the suture passing mechanism. At least one of the suture passing mechanism and the suture retrieving mechanism are configured to penetrate tissue, and one or the other of the suture passing mechanism and the suture retrieving mechanism are configured to move both axially and rotationally relative to the other. The proximal end of the elongated shaft comprises a handle for manipulating the distal end of the elongated shaft so that the suture passer is capable of delivering suture to a surgical site and retrieving suture from the surgical site, with the suture being passed through the tissue in the process.

In one preferred form of the invention, there is provided a suture placement system that serves as both an open or endoscopic surgical repair means wherein tissue defects can be externally ligated in close opposition by the steps of: passing a sharp penetrating needle through tissue, wherein the sharp penetrating needle carries a suture near the distal end of the needle; capturing a portion of the suture with a snare that is mounted to an axially and rotationally mobile armature that is carried by the needle; and withdrawing a portion of the suture which has been passed through the tissue which is to be repaired.

In another preferred form of the invention, there is provided a suture placement system that serves as both an open or endoscopic surgical repair means wherein tissue defects can be externally ligated in close opposition by the steps of: passing a sharp penetrating needle comprising a shaft and handle through tissue, wherein the sharp penetrating needle carries a suture snare near the distal end of the needle; positioning a suture adjacent to the suture snare using a cannula that is mounted to an axially and rotationally mobile armature that is carried by the needle; capturing a portion of the suture with the suture snare; and withdrawing a portion of the suture through the tissue which is to be repaired.

In one preferred form of the invention, the suture retrieving mechanism can comprise a contact face that slides or rotates against the suture passer tip, and another contact face that separates suture from the tip and a recess that captures the suture and provides a pulley-like surface to reduce running friction during withdrawal of suture through tissue.

In one form of the present invention, there is provided a surgical suture passer comprising:

an elongated cannula having at least one sharp distal end comprising a suture passing element; and

a shaft assembly slidably and rotationally disposed along the elongated cannula, wherein the shaft assembly comprises an offset tip comprising a suture capturing element that moves from a first position to a second position so as to releasably capture a suture and draw it away from the suture passing element.

In another form of the present invention, there is provided a surgical suture passer comprising:

an elongated shaft assembly having a distal end and a proximal end;

a tip located at the distal end of the elongated shaft assembly and comprising a sharp tissue penetrating element and carrying a suture; and

a co-acting mobile shaft assembly mounted to the elongated shaft assembly for axial and rotational movement relative thereto, and comprising a distal tip having a suture capturing element.

In another form of the present invention, there is provided a system for passing suture, the system comprising:

a first tip for penetrating tissue, the first tip adapted to carry suture; and

a second tip axially and rotationally movable relative to the first tip and adapted to engage the suture and draw the suture away from the first tip.

In another form of the present invention, there is provided a method for surgically placing suture, the method comprising:

penetrating tissue with a first elongated sharp shaft carrying a suture;

advancing a second elongated co-acting shaft carrying a suture capturing element so as to contact a portion of the suture, the second elongated co-acting shaft being axially and rotationally movable relative to the first elongated sharp shaft;

capturing the suture with the suture capturing element; and

withdrawing the first elongated shaft and second elongated shaft away from the tissue leaving the suture running through the penetrated tissue.

In another form of the present invention, there is provided a surgical suture passer comprising:

an elongated cannula having at least one sharp distal end and comprising a suture capturing element; and

a shaft assembly slidably and rotationally disposed along the elongated cannula, wherein the shaft assembly comprises an offset tip comprising a suture passing element adapted to advance suture for retrieval by the suture capturing element.

In another form of the present invention, there is provided a surgical suture passer comprising:

an elongated shaft assembly having a distal end and a proximal end;

a tip located at the distal end of the elongated shaft assembly and comprising a sharp tissue penetrating element and a suture capturing element; and

a co-acting mobile shaft assembly mounted to the elongated shaft assembly for axial and rotational movement relative thereto, and comprising a distal tip having a suture passing element.

In another form of the present invention, there is provided a system for passing suture, the system comprising:

a first tip for penetrating tissue, the first tip adapted to retrieve suture; and

a second tip axially and rotationally movable relative to the first tip and adapted to pass suture to a position adjacent to the first tip.

In another form of the present invention, there is provided a method for surgically placing suture, the method comprising:

penetrating tissue with a first elongated sharp shaft carrying a suture capturing element;

advancing a second elongated co-acting shaft carrying a suture passing element so as to be oriented toward the suture capturing element, the second elongated co-acting shaft being axially and rotationally movable relative to the first elongated sharp shaft;

capturing the suture with the suture capturing element; and

withdrawing the first elongated shaft and second elongated shaft away from the tissue leaving the suture running through the penetrated tissue.

In another form of the present invention, there is provided a method for surgically placing suture, the method comprising:

penetrating tissue with a first elongated sharp shaft comprising a suture receiving means;

advancing a second elongated co-acting mobile shaft comprising a suture dispensing means;

receiving suture from the suture dispensing means in the suture receiving means; and

withdrawing both shafts away from the tissue, leaving the suture running through the penetrated tissue.

In another form of the present invention, there is provided a method for surgically placing suture, the method comprising:

penetrating tissue with a first elongated shaft and advancing a second elongated shaft so that it is adjacent to the first elongated shaft, wherein at least one of the first elongated shaft and the second elongated shaft comprises a suture dispensing means and the other of the first elongated shaft and the second elongated shaft comprises a suture receiving means;

receiving suture from the suture dispensing means in the suture receiving means; and

withdrawing both shafts away from the tissue, leaving the suture running through the penetrated tissue.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 is a schematic side view of a novel surgical suture passer formed in accordance with the present invention;

FIG. 2 is a schematic side view of the distal portion of the novel surgical suture passer shown in FIG. 1;

FIG. 3 is the opposing schematic side view to that of FIG. 2;

FIG. 4 is a schematic side view of the novel surgical suture passer of FIG. 1 prior to its positioning in the tissue;

FIG. 5 is a schematic side view of the novel surgical suture passer of FIG. 1 after its placement through the tissue;

FIG. 6 is a schematic side view of the novel surgical suture passer of FIG. 1 after the step of positioning the suture retrieval tip into contact with the suture;

FIG. 7 is a schematic side view of the novel surgical suture passer of FIG. 1, illustrating the step of snaring the suture with the suture retrieval tip;

FIGS. 8 and 9 are schematic side views of the novel surgical suture passer of FIG. 1, illustrating the steps of drawing suture out of the distal tip of the surgical suture passer and then away from the tissue;

FIG. 10 is a schematic side view of the novel surgical suture passer of FIG. 1, illustrating the step of withdrawing both the distal tip of the surgical suture passer and the suture retrieval tip away from the tissue and leaving suture extending therethrough;

FIG. 11 is a schematic view of an alternative embodiment of novel suture passer, wherein the suture retrieval tip is adapted to be passed through tissue;

FIG. 12 is a schematic view of the suture passer shown in FIG. 11 prior to positioning in the tissue;

FIG. 13 is a schematic view of the suture passer shown in FIG. 11, with the sharp distal tip of the surgical suture retriever penetrating through the tissue;

FIG. 14 is a schematic view of the suture passer shown in FIG. 11, with its suture-snaring wire loop deployed out of the sharp distal tip of the surgical suture retriever;

FIGS. 15 and 16 are schematic views of the suture passer shown in FIG. 11, illustrating the step of the suture being delivered from the suture delivery tip towards, and through, the suture-snaring wire loop;

FIG. 17 is a schematic view of the suture passer shown in FIG. 11, illustrating the step of the suture-snaring wire loop being retracted back into the sharp distal tip of the surgical suture retriever while the suture is supported by the suture delivery tip;

FIG. 18 is a schematic view of the suture passer shown in FIG. 11, illustrating the step of drawing suture out of the suture delivery tip and away from the tissue;

FIG. 19 is a schematic view of another embodiment of the novel suture passer of the present invention;

FIG. 20 is a schematic view of the suture passer shown in FIG. 19, illustrating the suture-snaring wire loop deployed from the distal tip and suture being delivered through a suture delivery shaft and tip;

FIGS. 21-23 are schematic views of still another alternative embodiment of the novel suture passer, utilizing a suture capture eyelet, whereby the eyelet is selectively opened or closed by changing the position of the eyelet relative to a surrounding cannula;

FIGS. 24A, 24B, 24C and 24D are schematic views of yet another alternative embodiment of the novel suture passer, utilizing a suture capture tip having a snap-fit notch;

FIG. 25 is a schematic view of another alternative embodiment of the novel suture passer, utilizing a distal tip, formed from a solid rod comprising a transverse or angled eyelet, for capturing suture—suture can be free to slide through the eyelet, and/or to maintain a low streamlined profile, and/or the suture can be permanently affixed by swaging, hot melting a head, and/or various means of bonding;

FIGS. 26-28 are schematic views of another alternative embodiment of the novel suture passer, utilizing a groove formed in the handle for guiding the movement of a secondary shaft relative to a primary shaft; and

FIG. 29 is a schematic view of an alternative embodiment of the novel suture passer utilizing a slidable portion/capturing element for capturing suture to the device.

While the above-identified drawings set forth the presently disclosed embodiments, other embodiments are also contemplated, as noted in the discussion. This disclosure presents illustrative embodiments by way of representation and not limitation. Numerous other modifications and embodiments can be devised by those skilled in the art which fall within the spirit and scope of the present invention.

### DETAILED DESCRIPTION OF THE PRESENT INVENTION

### First Construction

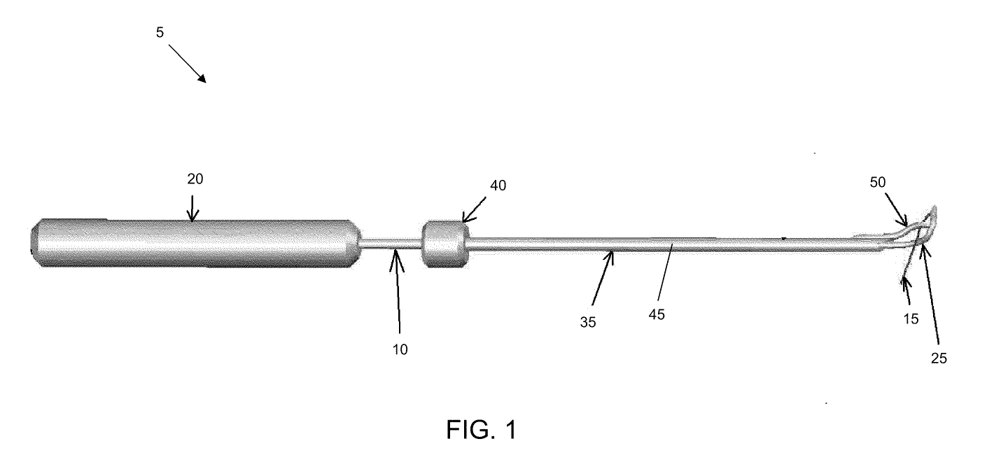

Looking first at FIGS. 1-3, there is shown a novel suture passer 5 for delivering suture to a surgical site, passing suture through tissue and retrieving suture from the far side of the tissue. In this construction, the suture passing means utilizes a sharp needle for passing the suture through tissue, and the suture retrieval means is configured so as to be axially and rotatably movable relative to the suture passing means, whereby to facilitate suture retrieval on the far side of the tissue.

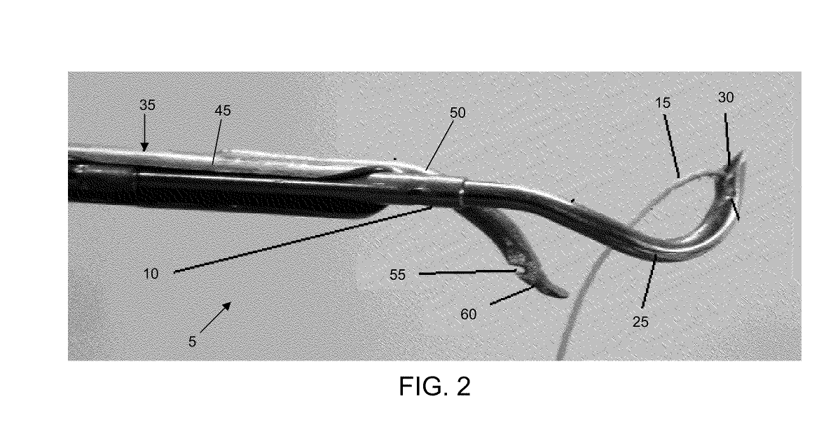

Suture passer 5 generally comprises an elongated cannula 10 for delivering a suture 15 to the surgical site, and a handle 20 for directing the suture passer. Elongated cannula 10 comprises a curved sharp distal tip 25 for penetrating tissue and a proximal end for mating with handle 20. Sharp distal tip 25 comprises a suture passing means (e.g., a lumen or opening) 30 in, or near, its distal tip (FIG. 2).

A slidable shaft assembly 35 is slidably disposed on elongated cannula 10. Slidable shaft assembly 35 comprises a positioning hub 40, a hollow shaft 45 for receiving elongated cannula 10, and a curved suture retrieval tip 50. Curved suture retrieval tip 50 in turn comprises a suture receiving means (e.g., a snaring notch) 55 (FIG. 2) adjacent to its distal end 60 for releasably capturing, and thereafter securing, suture 15 passed from distal tip 25 of elongated cannula 10, as will hereinafter be discussed in further detail. It should be appreciated that slidable shaft assembly 35 can be both (i) slid axially along elongated cannula 10, and (ii) selectively rotated about elongated cannula 10. Thus, it will be appreciated that suture receiving means 55 is configured to move both axially and rotationally relative to suture passing means 30.

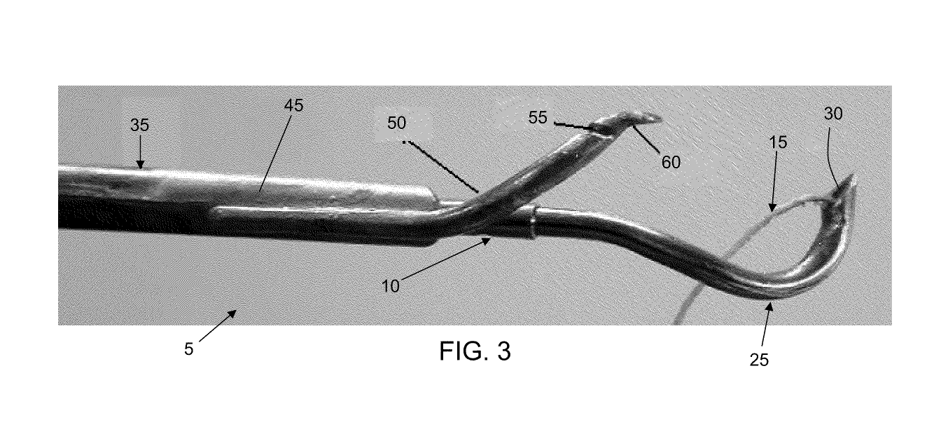

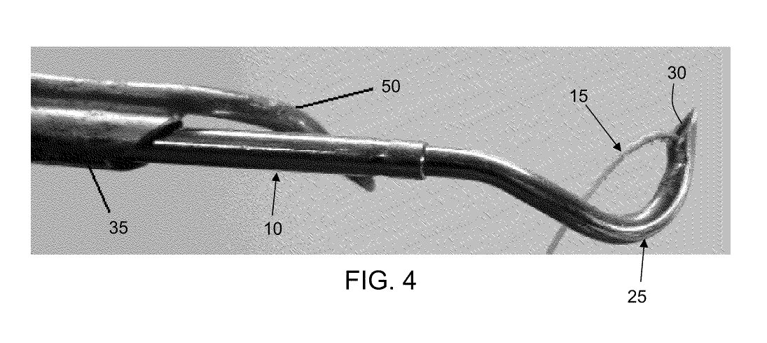

In use, and looking now at FIGS. 2-4, suture passer 5 is first loaded with a suture 15 therein, with a free end of the suture extending from suture passing means 30 in sharp distal tip 25. More particularly, a strand of suture 15 is disposed within the elongated cannula 10 so that one leg of the suture strand is internal to the cannula and a second leg of the suture strand exits suture passing means 30 and is external to the cannula. Then positioning hub 40 is slid proximally along elongated cannula 10 so as to draw suture retrieval tip 50 away from sharp distal tip 25, as shown in FIGS. 3 and 4. Suture passer 5 is then advanced to the surgical site.

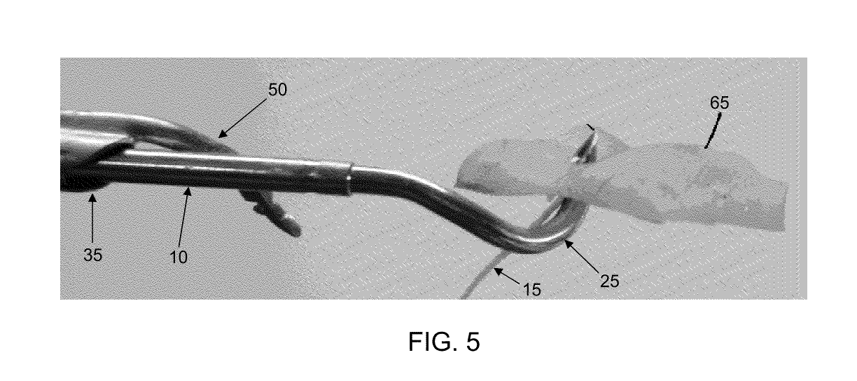

Once at the surgical site, and looking now at FIG. 5, suture passer 5 is moved so that its sharp distal tip 25 penetrates through tissue 65, carrying a portion of suture 15 through tissue 65, but leaving the free end of the suture extending back across to the near side of the tissue. This essentially erects a suture array on the far side of the tissue.

At this point, slidable shaft assembly 35, carrying suture retrieval tip 50 thereon, is slid distally along elongated cannula 10 so as to advance curved suture retrieval tip 50 to the far side of tissue 65 and position it adjacent to the aforementioned suture array located on the far side of tissue 65. If desired, slidable shaft assembly 35 may be rotated relative to elongated cannula 10 (and hence tissue 65) so as to facilitate positioning curved suture retrieval tip 50 at the desired location.

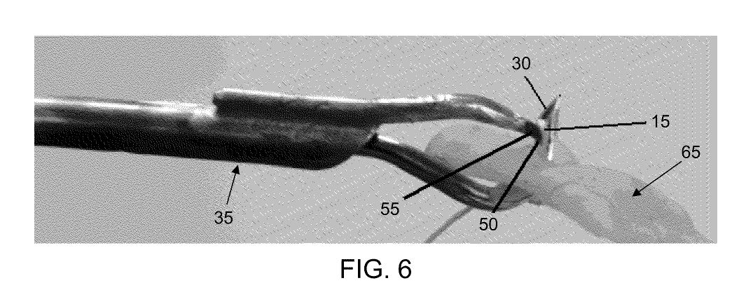

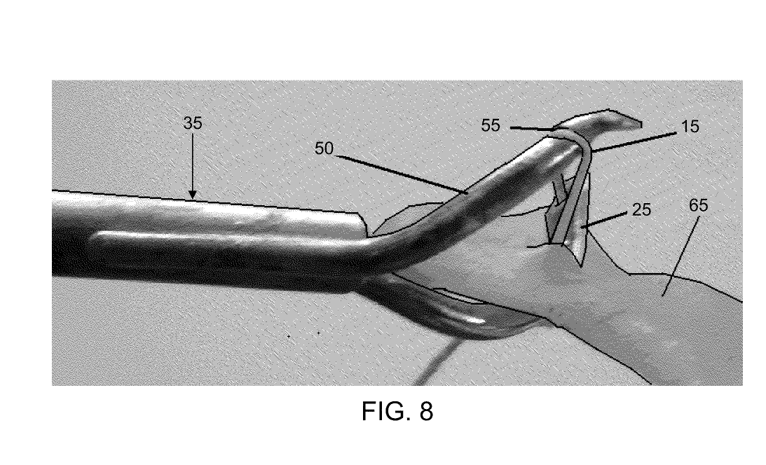

Looking next at FIGS. 6-8, suture retrieval tip 50 is advanced further distally so that it engages the suture emerging from suture passing means 30 of sharp distal tip 25. Snaring notch 55 of curved suture retrieval tip 50 is then used as a capturing means, by rotating slidable shaft assembly 35 via positioning hub 40, so that snaring notch 55 releasably engages suture 15 that is held in the suture array by sharp distal tip 25.

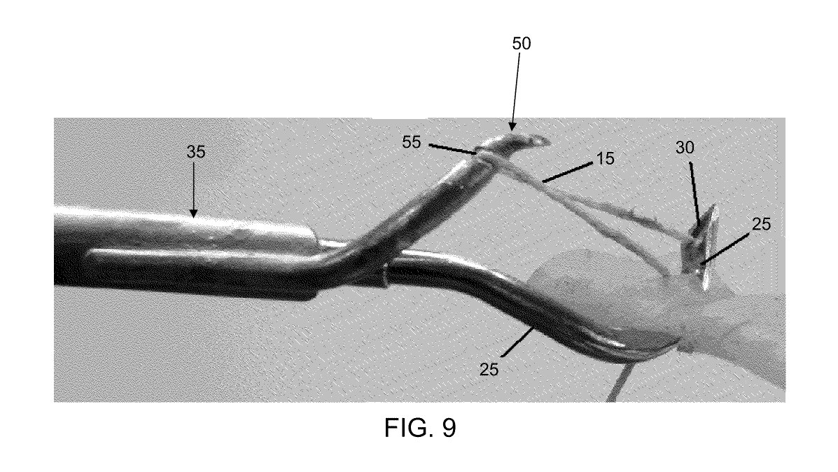

With the suture 15 engaged in snaring notch 55, and looking next at FIG. 9, curved suture retrieval tip 50 is drawn away from the tissue (via positioning hub 40), with suture 15 captured by snaring notch 55.

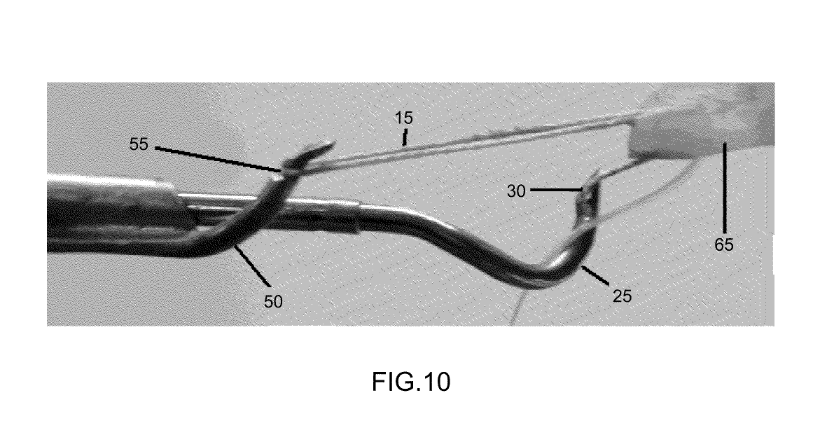

Looking next at FIG. 10, sharp distal tip 25 is then also withdrawn from the tissue that has been penetrated, leaving a loop of suture captured on snaring notch 55 and drawn through tissue 65.

This suture may then be used to approximate tissue, e.g., by tying a knot in the suture ends left extending from the tissue by any manner well known in the art.

It should be appreciated that the aforementioned snaring notch 55 may be replaced by any suture capturing feature or mechanism that allows suture to become attached to curved suture retrieval tip 50 through the interaction of curved suture retrieval tip 50 with the suture array, either alone or through interaction with both suture 15 and suture passing means 30 of sharp distal tip 25. By way of example but not limitation, such capturing features and mechanisms may include:

(i) an eyelet or notch formed in curved suture retrieval tip 50 designed with a snap-fit feature that allows a suture to slide into the eyelet or notch easily but that requires greater force to disengage the suture from the eyelet or notch;

(ii) a deformable portion of curved suture retrieval tip 50 that allows a suture to enter or exit into the deformable portion when in a first configuration but which prevents the suture from exiting when in a second configuration;

(iii) an eyelet or notch with a movable cover such that a suture may enter the eyelet or notch when the cover is open, but may not exit the eyelet or notch when the cover is partially or completely closed;

(iv) a feature on curved suture retrieval tip 50 that, when activated by contact with sharp distal tip 25, causes a notch or eyelet to open to an extent that allows a suture to enter but that, when disengaged from contact with sharp distal tip 25, causes the notch or eyelet to close, capturing the suture in the eyelet or notch; and

(v) a loop on a second tip, such as a loop of wire, that deforms when in contact with a suture or with sharp distal tip 25 so as to allow suture to enter the loop, after which the loop closes to capture the suture.

It should also be appreciated that other approaches for capturing suture utilizing a second tip may also be used.

To this end, the second tip for capturing suture may be provided by a second element comprising the second tip which is used in cooperation with a first element that comprises a first tip.

It will be appreciated that the second tip for capturing suture may be provided as an integral component of a single instrument comprising both the first sharp tip for advancing suture and the second tip comprising the suture capturing mechanism. By way of example but not limitation, the second suture capturing tip may comprise a second elongated portion comprising the suture capturing mechanism, whereby the second elongated portion is attached to a first elongated portion in a manner that allows the second elongated portion to move axially relative to the first elongated portion. Axial movement of the second elongated portion relative to the first elongated portion brings the suture capturing mechanism into contact with the suture, or into contact with both the suture and the first tip, whereby capturing the suture using the suture capturing mechanism.

By way of further example but not limitation, the second suture capturing tip may be provided as a component of an instrument comprising both a first tip for advancing suture and a second suture capturing tip. The second suture capturing tip may comprise a second elongated portion comprising a suture capturing means whereby the second elongated portion is attached to the first elongated portion in a manner that allows the second elongated portion to move both axially and rotationally relative to the first elongated portion. Axial and rotational movement, or isolated axial movement, or isolated rotational movement, of the second elongated portion relative to the first elongated portion brings the suture capturing mechanism into contact with the suture, or into contact with both the suture and the first tip, so that the suture is captured by the suture capturing means.

Therefore, and according to aspects illustrated herein, in one form of the invention there is provided a novel method for surgical suture placement comprising the steps of: (i) penetrating tissue with an elongated, substantially-sharp primary tip carrying suture so that the primary tip emerges on the far side of the tissue, exposing the suture and holding the suture in an array where the tissue that has been penetrated and the suture attachment or carry point at the sharp primary tip holds the suture in an array; and (ii) advancing an elongated secondary co-acting shaft along the same shaft which carries the primary tip, the shaft comprising a secondary optionally curved tip having suture receiving means disposed thereon, extending over the penetrated tissue, the secondary tip being slidably and rotationally positioned so as to (a) optionally manipulate, or pierce, tissue so as to assist penetration and exposure of the primary tip; (b) navigate past tissue; (c) contact the sharp primary tip and suture array (or to contact the suture array without contacting the primary tip); (d) further contactingly advance the secondary curved tip against the suture; (e) move the secondary curved tip so as to capture a portion of the suture to the suture receiving means in the secondary tip; (f) retract the secondary curved tip, drawing a loop of captured suture from the primary tip; and (g) retract the primary tip from the penetrated tissue, leaving the loop of suture passing through the tissue, and then removing the instrument from the surgical site, leaving the loop of suture passing through the tissue, then optionally withdrawing one leg of the suture loop from the body so as to leave a single strand of suture. The preceding steps may be followed by tying a knot in the suture (or by formation of a ligature or by reloading the suture in this or another instrument for further ligation).

### Second Construction

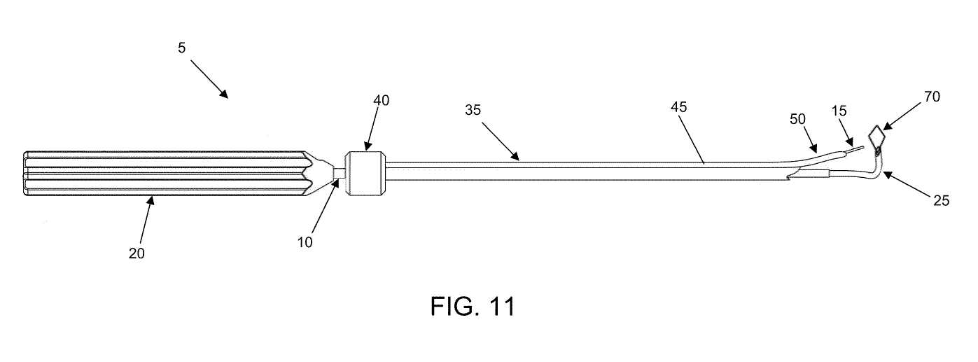

Looking next at FIGS. 11-18, there is shown an alternative novel suture passer 5 also formed in accordance with the present invention, but using an alternative suture delivery mechanism and suture capturing mechanism. More particularly, and in contrast to the approach used in the suture passer 5 shown in FIGS. 1-10, with the suture passer 5 of FIGS. 11-18, suture 15 is retrieved via a suture capturing mechanism 70 on sharp distal tip 25, and suture 15 is passed from a hollow suture delivery tip 50 of slidable shaft assembly 35. In other words, in this form of the invention, the suture retrieval means utilizes a sharp needle for passing through tissue, and the suture passing means are configured so as to be axially and rotatably movable relative to the suture retrieval means, whereby to facilitate suture retrieval on the far side of the tissue.

More particularly, suture passer 5 of FIGS. 11-18 generally comprises an elongated cannula 10 and a handle 20 for directing suture passer 5. Elongated cannula 10 is characterized by a sharp distal tip 25 for penetrating tissue and a proximal end for mating with handle 20. However, unlike the sharp distal tip 25 of the suture passer 5 of FIGS. 1-10, the sharp distal tip 25 of suture passer 5 shown in FIGS. 11-18 carries a suture capturing mechanism 70 therein for retrieving suture from the far side of tissue, as will hereinafter be discussed in further detail.

A slidable shaft assembly 35 is slidably disposed along elongated cannula 10. Slidable shaft assembly 35 comprises a positioning hub 40, a suture delivery tip 50, and a shaft 45 extending therebetween. As with suture passer 5 of FIGS. 1-10, it should be appreciated that slidable shaft assembly 35 can be both (i) slid axially along elongated cannula 10, and (ii) selectively rotated about elongated cannula 10, whereby the suture passing means of hollow suture delivery tip 50 may be axially and rotatably movable relative to the suture capturing mechanism 70 of sharp distal tip 25.

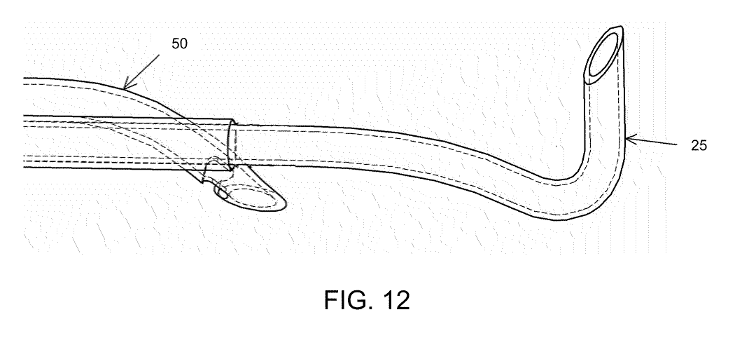

In use, positioning hub 40 is first maneuvered so as to retract and rotate suture delivery tip 50 so that suture delivery tip 50 is spaced from sharp distal tip 25. See FIG. 12.

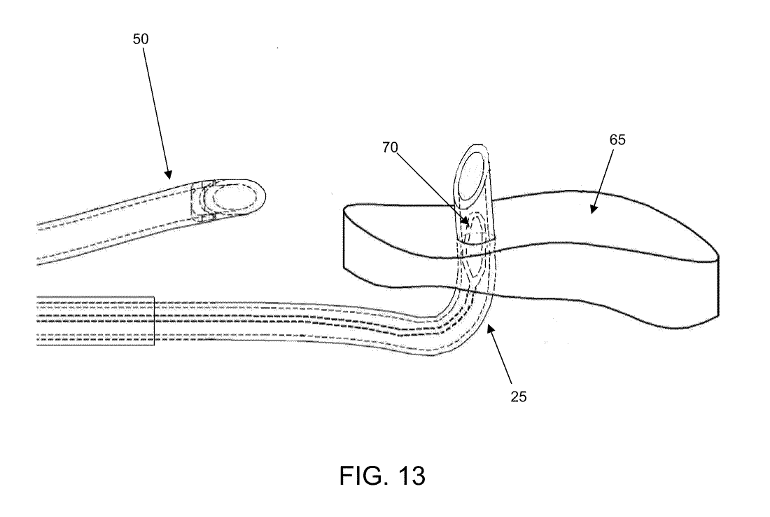

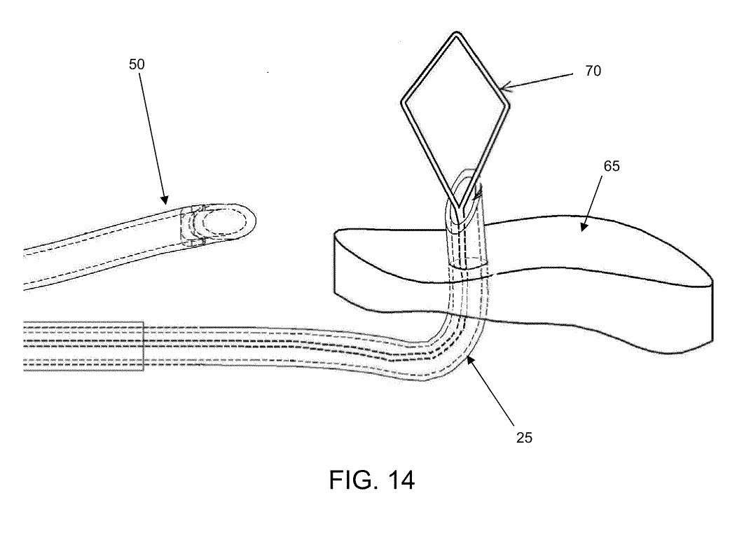

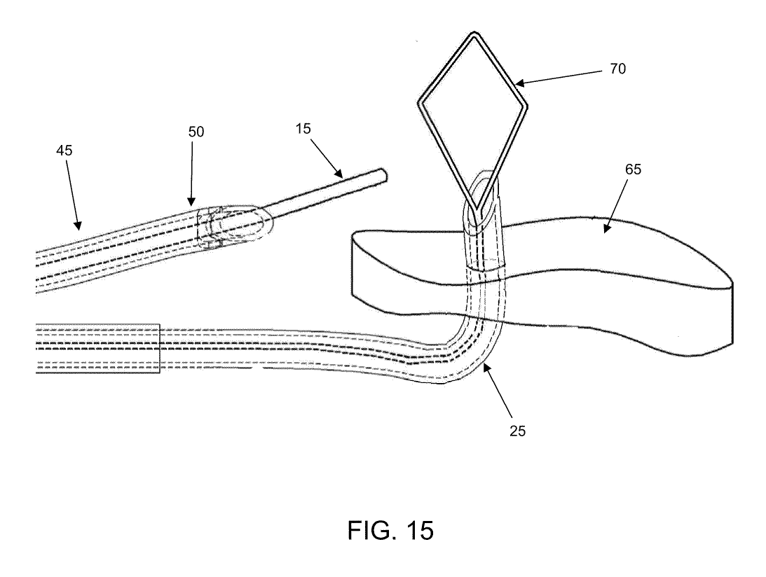

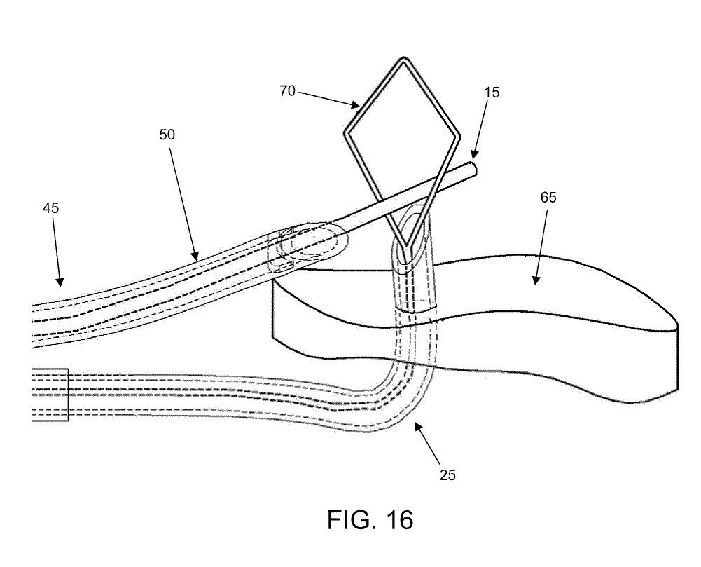

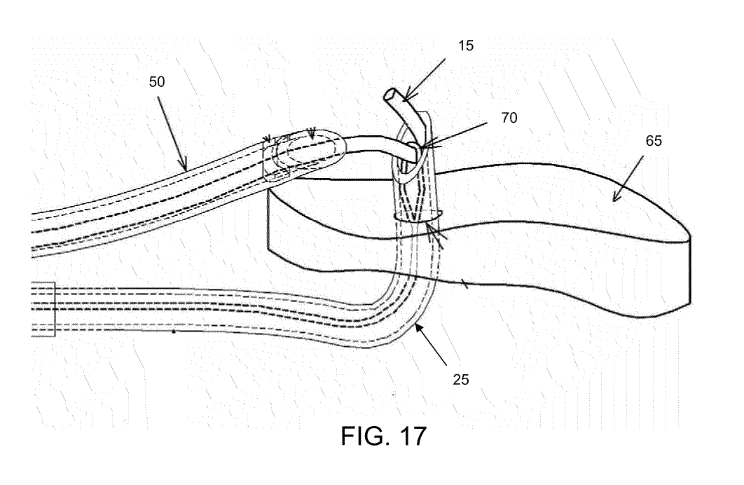

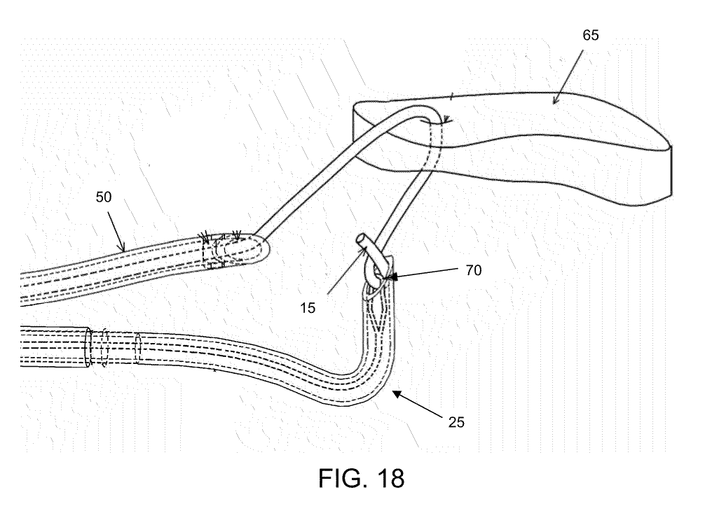

Then, and looking now at FIG. 13, sharp distal tip 25 is advanced to tissue 65. Sharp distal tip 25 then penetrates through tissue 65 from a first side to a second side, with suture capturing mechanism 70 positioned inside of sharp distal tip 25. Once sharp distal tip 25 has penetrated to the far side of tissue 65 (FIG. 13), suture capturing mechanism 70 is projected out of sharp distal tip 25 (FIG. 14). Suture delivery tip 50 is then positioned (via positioning hub 40) adjacent to suture capturing mechanism 70 (FIG. 15), moving axially and/or rotationally as needed, and then suture 15 is passed through suture capturing mechanism 70 (FIG. 16). Suture capturing mechanism 70 is then refracted slightly within sharp distal tip 25 (FIG. 17). With suture 15 held via suture capturing mechanism 70, suture delivery tip 50 is then withdrawn away from tissue 65 (FIG. 18), leaving a loop of suture in place and drawn through the tissue. The suture may then be used to approximate tissue, e.g., by tying a knot in the suture ends left extending from the tissue by any manner well known in the art.

Therefore, and according to aspects illustrated herein, in a second preferred form of the invention, there is provided a method for surgical suture placement comprising: penetrating tissue with an elongated, substantially-sharp primary tip that carries a suture receiver, so that the primary tip emerges from the far side of the tissue, exposing the suture receiver; placing the suture into the suture receiver with a co-acting secondary mobile element; withdrawing the suture though tissue with the suture receiver; and removing the instrument from the surgical site, leaving suture placed in or around the tissue. The preceding steps may be followed by tying a knot in the suture.

The steps of application of the present invention may optionally be altered by the user to utilize the secondary tip (i.e., the mobile element) to apply pressure to the tissue to be penetrated, so as to encourage the penetration and adequate emergence of the primary tip. In other words, suture delivery tip 50 may be positioned against the back side of tissue 65 so as to stabilize the tissue as sharp distal tip 25 is advanced through the tissue.

### Third Construction

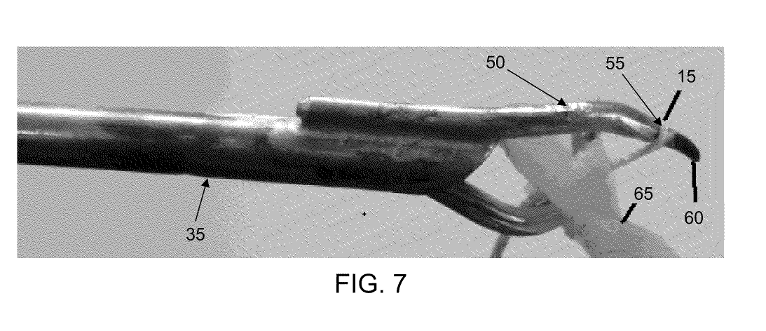

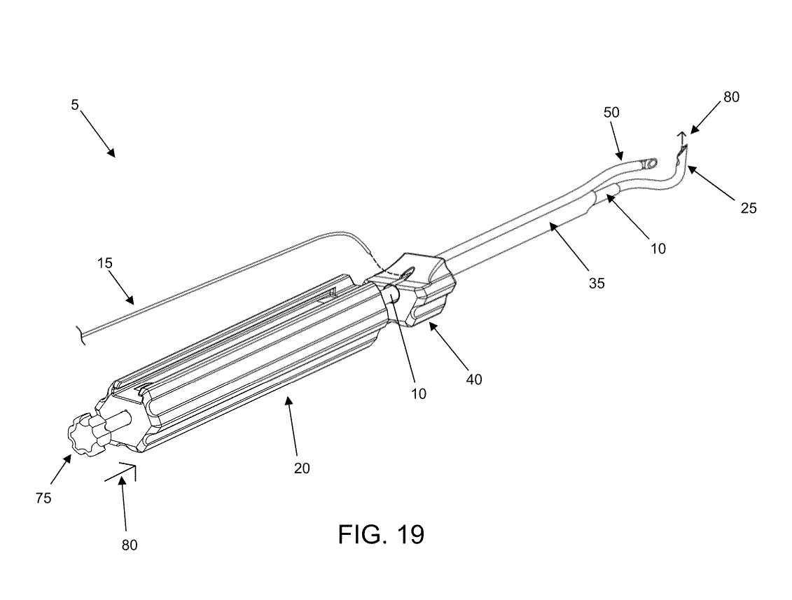

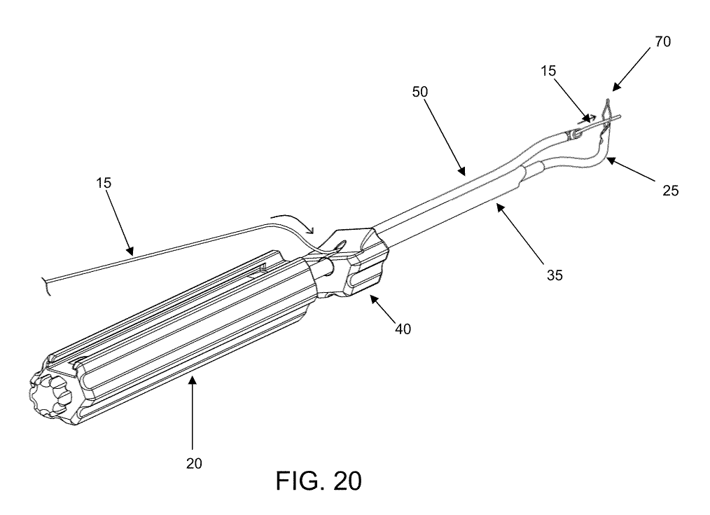

In a further embodiment of the present invention, and looking next at FIGS. 19 and 20, there is provided another suture passer 5. Suture passer 5 of FIGS. 19 and 20 is generally similar to the suture passer 5 of FIGS. 11-18. More particularly, suture passer 5 of FIGS. 19 and 20 generally comprises an elongated cannula 10 terminating in a sharp cannulated tip 25 for penetrating tissue. Cannulated tip 25 provides a passage through which a suture retrieving device 70 may be passed, so that it exits out of sharp cannulated tip 25. For purposes of illustration, suture retrieving device 70 is shown as a wire loop, commonly known as a suture lasso, but it should be appreciated that suture retrieving device 70 may comprise any other appropriate capturing mechanism, including a trailer strand of suture as shown in FIG. 7. Suture passer 5 also comprises a hollow suture delivery tip 50 which is mounted to elongated hollow cannula 10 via a slidable shaft assembly 35. A hub 40 is used to axially and rotationally position hollow suture delivery tip 50 relative to sharp cannulated tip 25 in the manner previously discussed.

In use, and looking now at FIG. 19, suture 15 is inserted into the proximal end of hollow suture delivery tip 50 and passed to its distal end. Control knob 75, at the proximal end of handle 20, is pulled back to retract suture retrieving device 70 into cannulated tip 25.

Once sharp tip 25 has penetrated through the tissue, control knob 75 is moved forward, in the direction of arrow 80, so as to cause suture receiving device 70 to be deployed from cannulated tip 25 in the direction of arrow 80.

Looking next at FIG. 20, with sharp cannulated tip 25 passed through the tissue, suture delivery tip 50 (with suture 15 extending therefrom) is moved (via hub 40) so that its distal end is adjacent sharp cannulated tip 25. Suture 15 and/or suture delivery tip 50 are then manipulated as needed so as to position suture 15 extending therefrom in contact with suture retrieving device 70 (or, alternatively, suture delivery tip 50 is positioned so that suture retrieving device 70 may be manipulated to capture suture 15 extending from suture delivery tip 50). Once suture 15 is captured by suture retrieving device 70, suture retrieving device 70 is withdrawn and retracted into sharp cannulated tip 25, carrying the suture therewith. The foregoing steps may be followed by tying a knot in the suture ends left extending from the tissue by any manner well known in the art.

Alternatively, suture retrieving device 70 may capture and carry suture 15 without being fully withdrawn and refracted completely into the cannulated tip 25. Cannulated tip 25 and suture delivery tip 50 may then be withdrawn from the tissue, leaving a loop of suture passing through the tissue. Again, the foregoing steps may be followed by tying a knot in the suture ends left extending from the tissue by any manner well known in the art.

### Additional Constructions

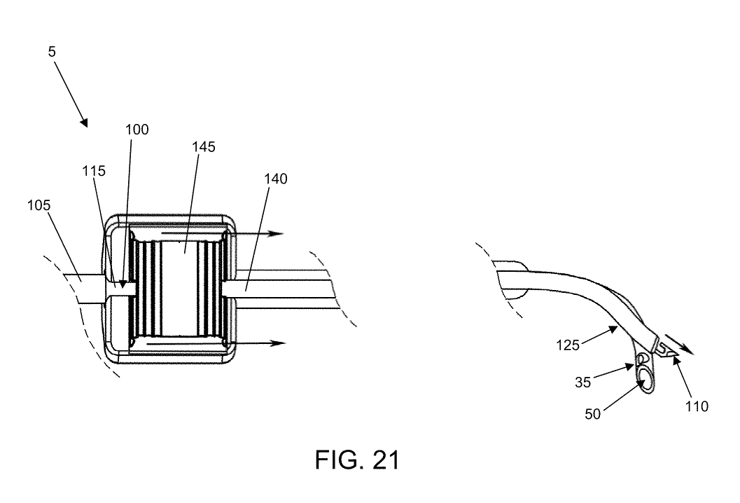

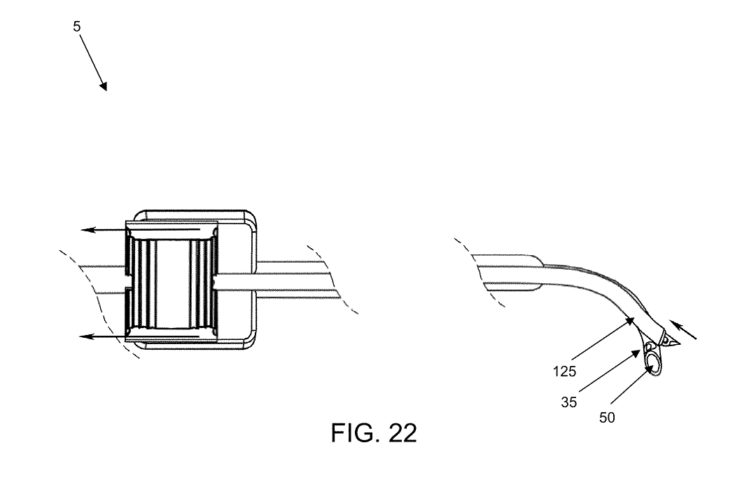

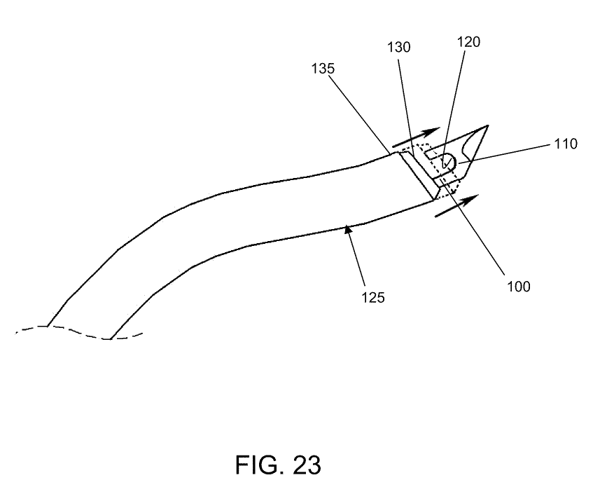

Looking next at FIGS. 21-23, there is shown an alternative construction for the novel suture passer of the present invention. In this embodiment, suture passer 5 generally comprises suture grasping means and suture passing means.

The suture grasping means generally comprises an elongated rod 100 having a handle 105 (or other operator interface). Elongated rod 100 comprises a curved, sharp distal tip 110 suitable for penetration of soft tissue, and a proximal end 115 suitable for mating with handle 105. Distal tip 110 comprises a suture attachment mechanism 120 to hold a suture in close proximity to distal tip 110. Suture passer 5 also comprises an externally disposed, slidable and rotatable pre-bent tip and shaft 125 comprising a suture receiving means (e.g., opening) 130 in or near its distal tip 135. Shaft 125 is connected, at its proximal end 140, to positioning hub 145. Shaft 125 typically remains retracted relative to distal tip 110 during positioning or tissue penetration. Shaft 125 can be advanced to contact distal tip 110 (and the suture trailing from distal tip 110) so that the suture can be captured by suture receiving means 130.

In a manner similar to the construction shown in FIGS. 11-18, the suture passing means comprises a slidable shaft assembly 35, including a hollow suture delivery tip 50, which can be both (i) slid axially along shaft 125, and (ii) selectively rotated about shaft 125.

Thus, in this construction, the suture passer comprises an externally disposed slidable, or slidable and rotatable, shaft 125 with a positioning hub 145 on the proximal portion and a suture attachment mechanism 120 on the distal portion whereby the suture attachment mechanism 120 has a closed position and an open position, and whereby positioning hub 145 comprises a control mechanism so that the suture attachment mechanism 120 can be changed from the open position to the closed position through manipulation of the positioning hub. The suture attachment mechanism 120 opened and closed positions may be effected by sliding a sleeve over a wire or solid rod, where the wire or solid rod comprises a bent or machined portion on the distal end forming a suture receiving notch or loop 120 and the sleeve moves in response to operator input through the positioning hub 145 to slide along the wire or solid rod, so that when the sleeve is slid distally, the sleeve blocks the entrance to the suture receiving notch or loop 120 of the wire or solid, in which configuration the suture attachment mechanism 120 is in the closed position and when the sleeve is refracted toward the proximal portion, the sleeve no longer blocks the suture receiving notch or loop 120 and the suture attachment mechanism is thus in the open position.

In a further embodiment of a shaft with a proximal positioning hub 145 and a distal suture attachment mechanism 120, a wire or solid shaft with a formed or machined suture receiving loop or notch 120 is disposed within a cannula that comprises an outer portion of the shaft and moved axially within the cannula in response to input from the operator through the positioning hub 145. When the wire or solid shaft is slid distally, the suture receiving notch or loop 120 is in an open position so that suture may enter the notch or loop 120 and when the wire or solid shaft is drawn back into the cannula, the suture receiving notch or loop 120 is in the closed position whereby the suture is prevented from leaving the suture receiving notch or loop 120.

Shaft 100 may comprise a suture attachment mechanism 120 that captures a suture in a snap-fit manner whereby the suture is captured by sliding into a slot or hole and whereby less force is required to slide the suture into the slot or hole than is required to slide the suture out of the slot or hole.

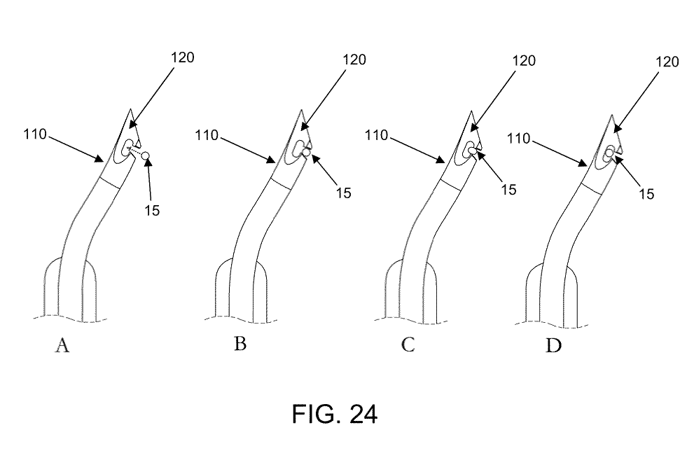

By way of example but not limitation, FIGS. 24A, 24B, 24C and 24D illustrate an embodiment of the present invention whereby the suture retention means is a snap-fit notch 120. The snap-fit notch 120 comprises an opening in the shaft into which suture may pass whereby a section of the opening is smaller than the diameter of the suture. The suture is shown in a cross-sectional view. In FIG. 24A, the suture is shown approaching the notch. A solid arrow indicates the motion of the suture relative to the notch. In FIG. 24B, the suture is shown just prior to entering the narrow portion of the notch. FIG. 24C illustrates the suture passing through the narrow portion of the notch. The suture has a diameter larger than the narrow portion of the notch which requires that the diameter of the suture deform in order for the suture to pass through this narrow portion. The suture cross-section is deformed in FIG. 24C. Force is required to deform the suture and allow it to pass through the narrow portion of the notch and into the open area of the notch. When the suture is in the open area of the notch, as shown in FIG. 24D, additional force is required to deform the suture and move it back through the narrow portion of the notch so the suture is effectively captured in the open portion until such force is applied.

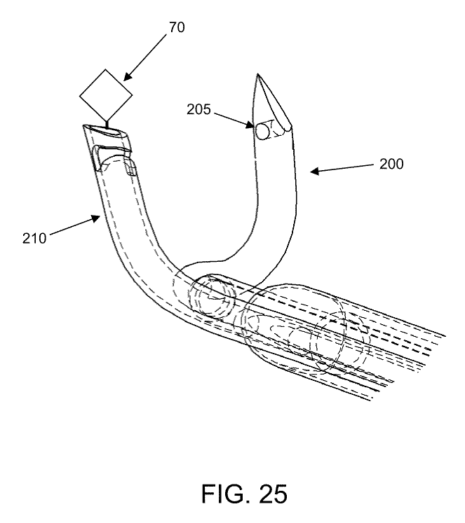

FIG. 25 is an embodiment of the present invention showing a first tip 200 that is formed from a solid rod and which comprises an eyelet 205 for holding suture. Suture may pass through the eyelet 205 on the tip of the solid rod so that it can be held by the eyelet in the solid rod and captured by a suture retrieving means 70 on the second tip 210.

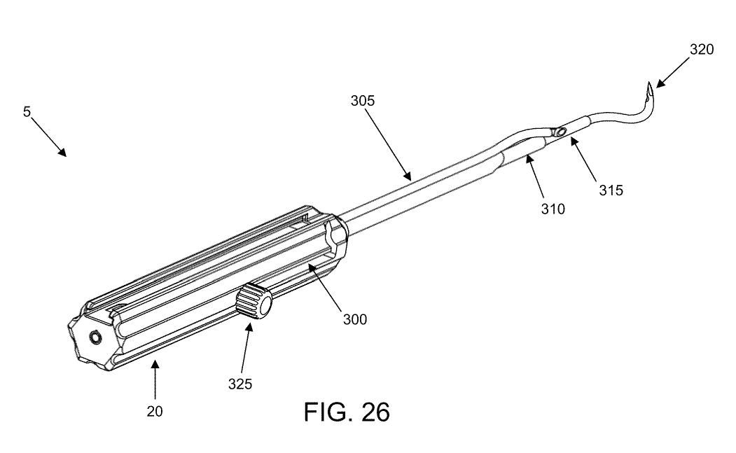

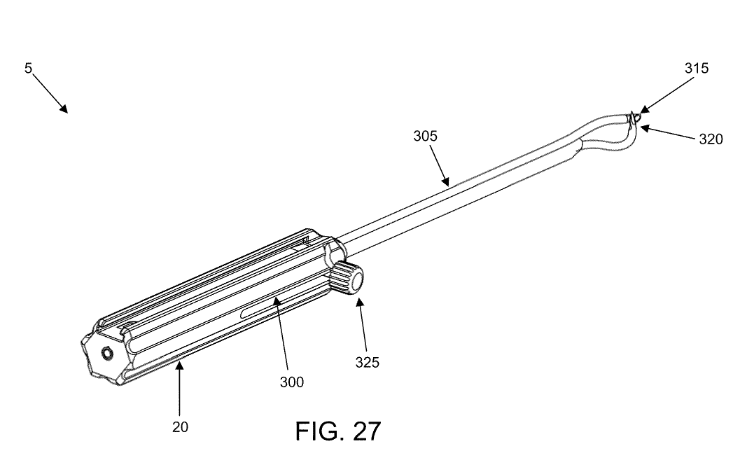

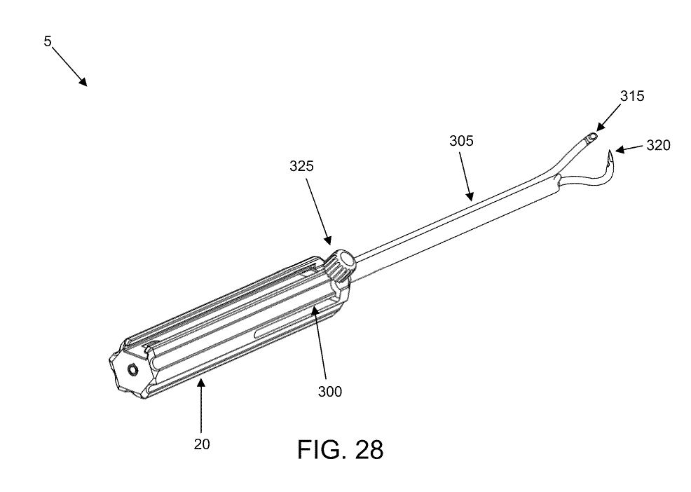

FIGS. 26-28 illustrate an embodiment of the present invention showing a guiding groove 300 in the handle 20 that guides the movement of the second shaft 305 relative to the first shaft 310. The groove 300 guides and limits the motion of the second tip 315 relative to the first tip 320. The motion of the second tip 315 is actuated by movement of the control knob 325. FIG. 26 shows the second shaft 305 fully retracted. FIG. 27 shows the second shaft 305 projected forward. FIG. 28 shows the second shaft 305 rotated.

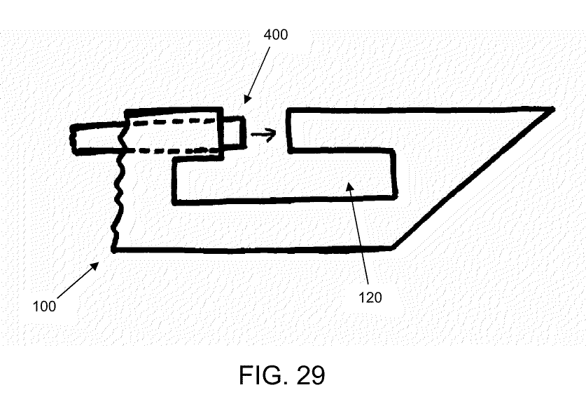

Shaft 100 may comprise a suture attachment mechanism 120 with a slidable portion/capturing element 400 whereby the suture is captured when the slidable portion 400 is advanced and is released when the slidable portion is withdrawn. See FIG. 29.

### Modifications

It is to be understood that the present invention is by no means limited to the particular constructions herein disclosed and/or shown in the drawings, but also comprises any modifications or equivalents within the scope of the invention.

What is claimed is:

1. A surgical suture passer comprising:
an elongated cannula having at least one sharp distal end comprising a suture passing element; and
a shaft assembly slidably and rotationally disposed along the elongated cannula, wherein the shaft assembly comprises an offset tip comprising a suture capturing element that moves from a first position to a second position so as to releasably capture a suture and draw it away from the suture passing element.

2. A surgical suture passer comprising:
an elongated shaft assembly having a distal end and a proximal end;
a tip located at the distal end of the elongated shaft assembly and comprising a sharp tissue penetrating element and carrying a suture; and
a co-acting mobile shaft assembly mounted to the elongated shaft assembly for axial and rotational movement relative thereto, and comprising a distal tip having a suture capturing element.

3. A system for passing suture, the system comprising:
a first tip for penetrating tissue, the first tip adapted to carry suture; and
a second tip axially and rotationally movable relative to the first tip and adapted to engage the suture and draw the suture away from the first tip.

4. A method for surgically placing suture, the method comprising:
penetrating tissue with a first elongated sharp shaft carrying a suture;
advancing a second elongated co-acting shaft carrying a suture capturing element so as to contact a portion of the suture, the second elongated co-acting shaft being axially and rotationally movable relative to the first elongated sharp shaft;
capturing the suture with the suture capturing element; and
withdrawing the first elongated shaft and second elongated shaft away from the tissue leaving the suture running through the penetrated tissue.

5. A surgical suture passer comprising:
an elongated cannula having at least one sharp distal end and comprising a suture capturing element; and
a shaft assembly slidably and rotationally disposed along the elongated cannula, wherein the shaft assembly comprises an offset tip comprising a suture passing element adapted to advance suture for retrieval by the suture capturing element.

6. A surgical suture passer comprising:
an elongated shaft assembly having a distal end and a proximal end;
a tip located at the distal end of the elongated shaft assembly and comprising a sharp tissue penetrating element and a suture capturing element; and
a co-acting mobile shaft assembly mounted to the elongated shaft assembly for axial and rotational movement relative thereto, and comprising a distal tip having a suture passing element.

7. A system for passing suture, the system comprising:
a first tip for penetrating tissue, the first tip adapted to retrieve suture; and
a second tip axially and rotationally movable relative to the first tip and adapted to pass suture to a position adjacent to the first tip.

8. A method for surgically placing suture, the method comprising:
penetrating tissue with a first elongated sharp shaft carrying a suture capturing element;
advancing a second elongated co-acting shaft carrying a suture passing element so as to be oriented toward the suture capturing element, the second elongated co-acting shaft being axially and rotationally movable relative to the first elongated sharp shaft;
capturing the suture with the suture capturing element; and
withdrawing the first elongated shaft and second elongated shaft away from the tissue leaving the suture running through the penetrated tissue.

9. A method for surgically placing suture, the method comprising:
penetrating tissue with a first elongated sharp shaft comprising a suture receiving means;
advancing a second elongated co-acting mobile shaft comprising a suture dispensing means;
receiving suture from the suture dispensing means in the suture receiving means; and
withdrawing both shafts away from the tissue, leaving the suture running through the penetrated tissue.

10. A method for surgically placing suture, the method comprising:
penetrating tissue with a first elongated shaft and advancing a second elongated shaft so that it is adjacent to the first elongated shaft, wherein at least one of the first elongated shaft and the second elongated shaft comprises a suture dispensing means and the other of the first elongated shaft and the second elongated shaft comprises a suture receiving means;
receiving suture from the suture dispensing means in the suture receiving means; and
withdrawing both shafts away from the tissue, leaving the suture running through the penetrated tissue.
